(19) United States
(12) Patent Application Publication
(10) Pub. No.: US 2002/0055678 A1
(43) Pub. Date: May 9, 2002

(54) Electrode probe coil for MRI

(76) Inventors: Greig C. Scott, Palo Alto, CA (US); Garry E. Gold, Los Altos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/904,600

(22) Filed: Jul. 12, 2001

Related U.S. Application Data
(60) Provisional application No. 60/217,979, filed on Jul. 13, 2000.

(51) Int. Cl. A61B 5/05

(52) U.S. Cl. 600/423; 600/420

(57) ABSTRACT

A coil for magnetic resonance imaging includes at least two spaced electrodes positionable within an object in proximity to a region of interest with feed wires coupling the electrodes to a signal detector. The electrodes and feed wire cooperatively function with tissue and fluid of the object to form an RF signal detecting coil. The electrodes can be needles or rings around the circumference of a catheter or electrodes which extend from and are retractable within a catheter.

### CROSS-REFERENCES TO RELATED APPLICATIONS

This patent application claims priority from Provisional Application No. 60/217,979 filed Jul. 13, 2000, which is incorporated herein by reference.

### STATEMENT AS TO RIGHTS TO INVENTIONS MADE UNDER FEDERALLY SPONSORED RESEARCH AND DEVELOPMENT

The U.S. Government has rights in the disclosed invention pursuant to NIH Grant No. 003297 to Stanford University.

### BACKGROUND OF THE INVENTION

This invention relates generally to magnetic resonance imaging, and more particularly the invention relates to coils for detecting MRI signals emitted from excited nuclei in an object being imaged.

In MRI, an object to be imaged is placed in a static magnetic field which magnetically aligns nuclei in the object. An RF pulse is used to tip the nuclei out of alignment, and the tipped nuclei give up small signals as they realign with the static magnetic field. Coils are then used to detect the emitted magnetic resonance signals. External receiving coils have been used in detecting the signals, and surface coils have been placed on the object to obtain more localized signals. Recently, attempts have been made to detect MRI signals within the object by the use of intravascular catheter probes. FIG. 1 illustrates a loopless dipole antenna coil proposed by O. Ocale and E. Atalar for intravascular imaging. See MRM 37: 112-118 (1997), U.S. Pat. No. 5,928,145. The inner conductor 10 of a coaxial cable 12 extends from the cable as a signal detector. Other prior art detectors have employed closed loop coils, whose sensitivity dies off within a few diameters of the coils. Also, closed loop coils require large catheters for intravascular use.

While magnetic resonance imaging is the most sensitive and accurate imaging technique available for assessment of articular cartilage and osteoarthritis, conventional MRI is limited to making static images of structures which are not in motion, with little access for intervention. A recent development of open MRI scanners such as the open GE 0.5T Signa at the Stanford Hospital allows physicians to perform procedures under MR guidance. The signal to noise and imaging speed of these open scanners is typically poor compared with conventional MRI, thus limiting the visibility of articular cartilage and osteoarthritis. To improve image quality and visibility of cartilage on these systems, physicians have turned to MR arthrography, where a dilute mixture of Gadolinium contrast agent is injected into the joint prior to imaging.

The present invention is directed to electrode probes which are readily employed in imaging confined areas and which provide higher sensitivity.

### BRIEF SUMMARY OF THE INVENTION

In accordance with the invention, two or more probes cooperatively function with tissue or fluid being imaged to effectively form a coil for detecting magnetic resonance signals emitted from the tissue or fluid.

In one embodiment, two electrodes are implanted in tissue with the tissue between the electrodes forming a parallel resistor-capacitor circuit that effectively closes the loop formed by the electrodes and feed wires to the electrodes. Alternatively, one electrode can be on the surface of the tissue. The impedance of the loop is matched to a preamplifier with the loop detecting MRI signals within the loop.

In other embodiments, the probes can be in RF ablation catheters or electrical stems implanted in a patient. The ablation electrodes can be the MRI detection electrodes.

The invention and objects and features thereof; will be more readily apparent from the following detailed description and appended claims when taken with the drawings.

### BRIEF DESCRIPTION OF THE DRAWINGS

FIG. 1 illustrates a prior art loopless MRI detection probe.

FIG. 2 illustrates one embodiment of an electrode probe in accordance with the invention.

FIGS. 3A-3F illustrate sensitivity of the probe of FIG. 2 in several planes and orientation of the probe with respect to the static magnetic field.

FIG. 4 illustrates an image created from data acquired with a probe in accordance with the invention.

FIG. 5 illustrates placement of an electrode probe in accordance with the invention intra-articularly in a patient with a defect in the patellar cartilage.

FIG. 6 is a plot of signal to noise ratio for a conventional surface coil and for a probe as illustrated in FIG. 2.

FIGS. 7A, 7B illustrate other embodiments of the invention.

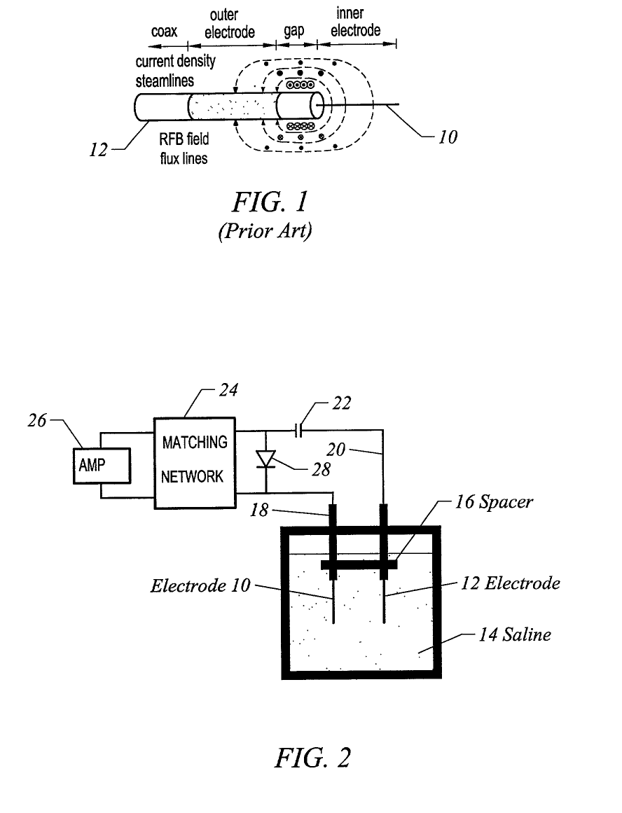

### DETAILED DESCRIPTION OF THE INVENTION

FIG. 2 illustrates an electrode probe coil in accordance with one embodiment of the invention. In the simplest form, the probe comprises two electrodes 10, 12 placed in a conducting medium such as tissue or saline 14. A spacer 16 maintains the relative positioning of electrodes 10, 12, and feed wires 18, 20 connect the electrodes through a DC blocking capacitor 22 to an impedance matching network 24 and amplifier 26. Diode 28 is connected between feed wires 18, 20 to prevent overloading of the matching network and amplifier during application of RF excitation pulses to tissue under examination. Detected signals are thus limited to the standard voltage drop of the diode 28. Alternatively, a switch can be serially connected with diode 28 to disconnect the diode during signal detection.

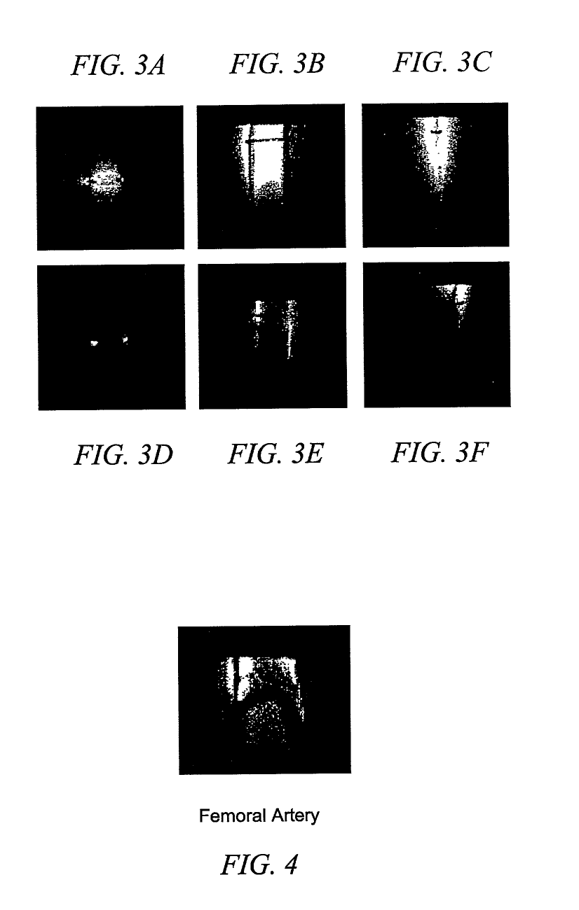

The sensitive imaging volume for the coil is located between the electrodes and the area enclosed by the corresponding feed wires. If the electrodes are positioned properly at either side of the region of interest, or a pattern of electrodes is used to surround the region of interest, the noise volume seen by the coil can be minimized. The pattern of sensitivity of the electrode probe coil also depends on the orientation of the coil with respect to the main magnetic field, B₀. The sensitivity pattern of the coil and its relationship to B₀ is shown in FIG. 3.

Referring to FIG. 3, FIGS. 3A-3C illustrate the sensitivity pattern in the axial, coronal, and sagittal planes with the electrodes in a plane parallel to B₀. Images 3D-3F illustrate the sensitivity pattern in the same planes with electrodes in a plane perpendicular to B₀.

The coil can be used to image excised specimens submerged in a saline bath. An example of this is shown in FIG. 4 which is an axial image of an excised human femoral artery using the electrode probe coil with a 1.5T MRI scanner. Any conductive medium can be used with the probe, such as human tissue which is largely normal saline. The electrode probe coil can be used intra-articularly in conjunction with MR arthrography or arthroscopy. Further, the probe can be used for guidance of therapy in an open MRI system, or for diagnosis or monitoring treatment in an open or conventional MRI system.

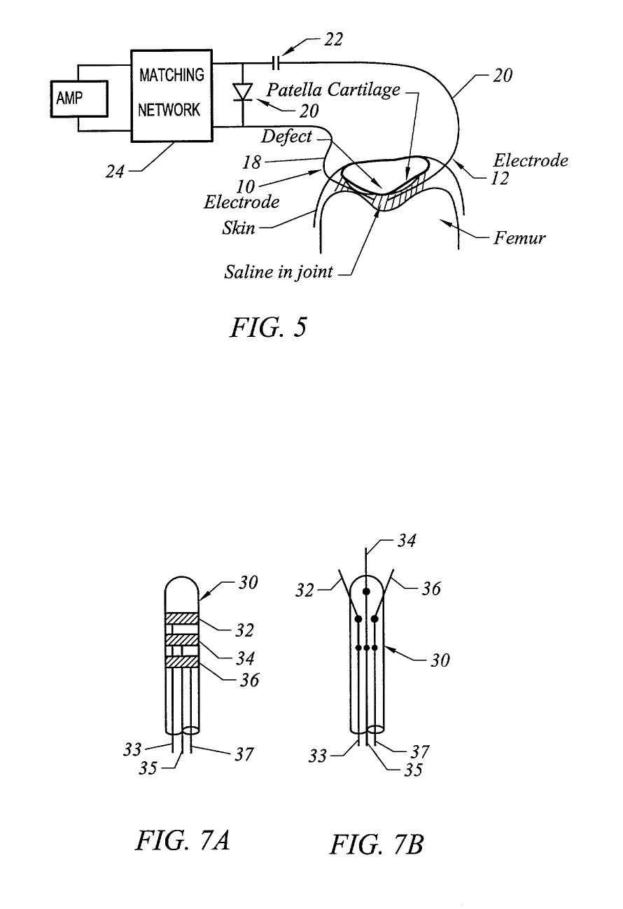

FIG. 5 is a schematic drawing of the placement of an electrode probe coil intra-articularly in a patient with a defect in the patellar cartilage. The joint is filled with saline, and the electrodes 10, 12 are placed near the defect to maximize signal to noise ratio in the area of interest. Again, feed wires 18, 20 connect electrodes 10, 12 through DC blocking capacitor 22 and matching network 24 to an amplifier 26.

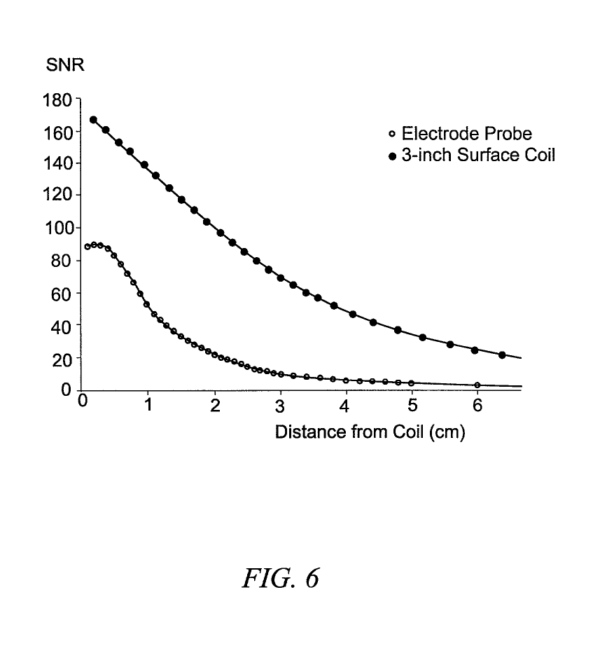

An MRI probe in accordance with the invention allows greater signal to noise ratio in detected signals within an object being imaged as compared to the use of a conventional surface coil. FIG. 6 is a graph illustrating curves of the signal to noise ratio versus distance from the coil for a three inch surface coil and for an electrode probe such as illustrated in FIG. 4. At the surface, the surface coil provides a SNR of approximately 170 which drops below 90 at a distance of between two and three centimeters from the surface. The electrode probe in accordance with the invention has a SNR of about 90 adjacent to the probe which drops off to a SNR of 50 at one centimeter from the probe. Thus is it seen that by placing a probe in accordance with the invention adjacent to tissue or fluid more than two centimeters from the surface of a patient, an improved SNR is realized for the detected signal.

FIGS. 7A and 7B illustrate other embodiments of a probe in accordance with the invention in which the electrodes are placed in or in conjunction with a catheter 30. In FIG. 7A the electrodes are conductive rings 32, 34, 36 around the circumference of catheter 30 which image tissue such as vascular wall. In FIG. 6B electrodes 32, 34, 36 are extendable from catheter 30 for obtaining MRI signals when catheter 30 is stationary. During movement of catheter 30 the electrodes are withdrawn to prevent obstruction of catheter movement within a blood vessel, for example.

Electrode probe coils in accordance with the invention provide improved MRI signals for tissue and fluid within an object being examined as opposed to the use of surface coils and other external coils. While the invention has been described with reference to specific embodiments, the description is illustrative of the invention and is not to be construed as limiting the invention. Various modifications and applications may occur to those skilled in the art without departing from the true spirit and scope of the invention as defined by the appended claims.

What is claimed is:

1. A probe for detecting magnetic resonance signals emitted from a region of interest in an object comprising:
(a) at least first and second electrodes positionable on or within the object in proximity to the region of interest, and
(b) feed wires coupling the electrodes to a signal detector,
wherein the electrodes and feed wires cooperatively function with matter within the region of interest to form a signal detecting coil.

2. The probe as defined by claim 1 wherein the first and second electrodes are spaced apart with matter within the region of interest therebetween.

3. The probe as defined by claim 2 wherein the matter comprises tissue.

4. The probe as defined by claim 2 wherein the matter comprises fluid.

5. The probe as defined by claim 2 wherein the number of electrodes exceeds two.

6. The probe as defined by claim 5 wherein the electrodes are carried by a catheter.

7. The probe as defined by claim 6 wherein electrodes are rings around the circumference of the catheter.

8. The probe as defined by claim 6 wherein the electrodes are extendable from and retractable within the catheter.

9. The probe as defined by claim 2 wherein the electrodes are carried by a catheter.

10. The probe as defined by claim 9 wherein the electrodes are rings around the circumference of the catheter.

11. The probe as defined by claim 9 wherein the electrodes are extendable from and retractable within the catheter.

12. The probe as defined by claim 2 wherein the electrodes comprise needles.

13. A method of imaging a region of interest in an object comprising the steps of:
(a) placing the object in a static magnetic field,
(b) applying RF excitation pulses to the region of interest, and
(c) detecting magnetic resonance signals from the region of interest with an array of at least two spaced electrodes in proximity to the region of interest.

14. The method as defined by claim 13 wherein the electrodes and feed wires to the electrodes cooperatively function with tissue in the region of interest to form an RF signal detecting coil.

15. The method as defined by claim 13 wherein the electrodes comprise needles.

16. The method as defined by claim 13 wherein the electrodes are carried by a catheter.

17. The method as defined by claim 16 wherein the electrodes comprise rings around the circumference of the catheter.

18. The method as defined by claim 16 wherein the electrodes are extendable from and retractable within the catheter.
